(19) United States
(12) Patent Application Publication
(10) Pub. No.: US 2007/0003591 A1
(43) Pub. Date: Jan. 4, 2007

(54) USE OF A PROPOLIS AS A COATING MATERIAL FOR MEDICAL IMPLANTS

(76) Inventors: Rudolf Hesselbarth, Albbruck (DE); Roland Rohde, Burgdorf (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 11/426,200

(22) Filed: Jun. 23, 2006

(30) Foreign Application Priority Data
Jun. 30, 2005 (DE) 10 2005 031 361.2

(51) Int. Cl. A61K 35/64; A61F 2/06

(52) U.S. Cl. 424/423; 424/539; 623/1.11

(57) ABSTRACT

Propolis as a coating material for medical implants. It also relates to the application of propolis, particularly of the constituent caffeic acid phenylethyl ester, for the manufacture of a drug for re-stenosis prophylaxis after percutaneous transluminar coronary angioplasty (PTCA) inhuman coronary arteries.

### FIELD OF THE INVENTION

The present invention relates to a new application of propolis as a coating material for medical implants. It also relates to the use of propolis, particularly the constituent caffeic acid phenylethyl ester for the manufacture of a drug for re-stenosis prophylaxis after percutaneous transluminar coronary angioplasty (PTCA) in coronary arteries.

### BACKGROUND OF THE INVENTION

Permanent medical implants, or implants designed for temporary residence in the body, require a high degree of biocompatibility of the materials used to minimise undesirable tissue reactions after implantation. Meanwhile a large number of biocompatible materials of the most varied types can be used. In the area of vessel supports (stents), for example, biocompatibility of the implant surface can be improved by coating the implant surface with silicon carbide or phosphoryl cholin, which reduces the thrombogenicity and any inflammatory reason on the implant surface.

Another problem area of modem implantation technology lies in the fact that the implant is repeatedly colonised by bacteria, where each colonisation does not necessarily result in an infection. The implant is first covered with the proteins, such as fibrin, fibronectin, albumin, laminin or vitronectin naturally occurring in the body to which bacteria (e.g., staphylococcus aureus) may adhere. The bacteria synthesise exopolysaccharides and form a biofilm on the surface of the implant. The heterogeneous adherent bacteria population of the biofilm are difficult to access the defence mechanisms of the body and for antibiotic therapies. Staphylococcus epidermis was isolated as the most common microorganisms in infections of implants, particularly intravascular catheter infections and pacemaker infections. Staphylococcus aureus and coagulase-negative staphylococci are further commonly occurring bacteria in implant infections. Such infections can frequently only be treated by explanation and renewed implantation, if necessary accompanied by systemic antibiotic administration. For example, the systemic administration of antibiotics with surgical treatment is recommended in the case of infections of pacemaker or pacemaker-defibrillator systems.

Since it is very difficult to penetrate the biofilm matrix with antibiotics and kill the cells associated with the biofilm, strategies must be developed which either specifically the polysaccharide matrix of the biofilm or prevent the adhesion of the biofilm and hence the formation of a biofilm. The latter alternative provides, for example, for the application of a coating on the implant, which either has an intrinsic antibiotic action or contains antibiotics. Heart valve sutures, for example, were coated with silver or catheters were coated with silver salts or antibiotics.

If the decision has been made for a coating system to improve biocompatibility and/or to reduce the risk of infection, the selection of a suitable coating material is anything but a trivial matter, for it should be possible, where feasible, to process the material easily with conventional production methods to allow the most uniform surface cover of the implant as possible. The material should obviously also be biocompatible and anti-infectious, available in sufficient quantities and, if possible, also economical of course. Merely finding a suitable material therefore requires a high degree of understanding of the underlying biological mechanisms, a knowledge of the required material properties, in terms of processing and subsequent use, and also a knowledge of the availability and possible costs associated with using the material. The location of such a material is therefore very expensive and cannot be carried out by a standardised manner for the very reason that many material properties that may be relevant to the intended purpose of use have not yet been described or are not predictable, and can only be documented after expensive tests have been carried.

Despite the enormous efforts that have been made in recent decades to improve the problems in implant technology discussed, and despite the progress made in establishing the underlying biological mechanism, constant further development and rediscovery of suitable materials and the use of complex coating systems to reduce rejection reactions and infections, there is still a considerable requirement for new approaches to solutions which improve the situation or at least offer alternatives to what is already known.

### SUMMARY OF THE INVENTION

A feature of the invention is to overcome the disadvantages of the state of the art described or at least provide a further alternative to known solutions.

This feature is achieved according to a first exemplary embodiment of the invention by the use of propolis as a coating material for medical implants.

Propolis is a dark yellowish to light brown, resin-like mass that softens between the fingers, with a spicy-balsam like odour and a melting temperature of between 50 and 70° C., which is collected by bees and is used in the bee hive as a covering for the walls and for securing the honeycombs (hive dross, plugging wax, bee glue, bee cement, bee resin). Propolis is a natural substance whose qualitative and quantitative composition varies considerably. The following constituents have already been isolated and described.
a) Amino acids;
   Propolis contains, in small quantities, pyroglutaminic acid, which probably derives from the bee metabolism.
b) Aliphatic acids and their esters;
   Long-chain acids (e.g. behenic, palmitic, stearic and myristinic acid) have been found in propolis, probably originating from beeswax. The origin of the short-chain unstable acids, such as succinic, angelic and butyric acid and their esters (e.g. isobutyric acid) is not known, but is probably vegetable.
c) Aromatic acids and their esters;
   The aromatic acids and their esters are in most cases of vegetable origin. Important examples are cinnamon acid, methyl salicylate, caffeic acid and their esters, vanillic acid and p-cumaric acid benzaldehyde.
d) Alcohols;
   In all probability some glycerine derivatives derive from the bee metabolism and glycerol is present in the wax. Other alcohols, e.g., hydroquinone and cinnamon alcohol, are also found in poplars.
e) Aldehydes;
   The major representatives of the aldehydes are vanillin, protocatechualdehyde(3,4-dihydroxybenzldehde), p-; hydroxybenzaldehyde.
f) Chalcones;
   Chalcones are among the flavonoids and accumulate as glycosides only in small plant families. The chalcone most commonly occurring in nature is butein (2′,3,4,4′-tetrahydroxychalcone).
g) Dihydrochalcones;
   Dihydrochalcones are flavanone glycosides and are characteristic, for example, of propolis balsamifera (section Tahamhaca). They are only present in propolis in small quantities.
h) Flavanones, flavanes and flavonols;
   Flavanones are the derivatives of flavane (phenyl-4H-chromane) associated with the phenolic compounds, they have an oxo group in the 4-position and they may also be regarded as hydrated flavones. Flavonols belong to the class of generally yellow, cream-coloured odourless and tasteless plant dyes that is included in the flavonoids, which share the basic structure of flavone, in the case of the 3-positon flavonols, a hydroxylated structure. The compounds represent a primary component of European propolis. The principal representatives are pinocembrin(5,7-dihydroxyflavanone), pinobanksin(3,5,7-trihydroxyflavanonol), naringenin, sacuranetin, galangin(3,5,7-trihydroxyflavone) and quercetin(3,3′4′5,7-pentahydroxyflavone).
i) Hydrocarbons, ketones, terpenoids and other components;
   Ketones, terpenoids and other components are only present in propolis in small quantities. The CIO-terpenoids are said to have a strong odour and they are probably responsible for the odour of propolis.

The considerable proportions of wax present in propolis were demonstrated in analyses in the form of its principal components as monoesters and hydrocarbons. The hydrocarbons consist of a complex mixture of n-alkanes which have an odd number of C atoms in the range of C₂₃-C₃₅. The most common found were C₂₇H₅₆, C₂₉H₆₀, C₃₁H₄₆ and C₃₃H₆₈.

Propolis for the purposes of the invention is standardised as follows: Propolis samples are collected from moderated climatic zones, preferably propolis samples from poplars of section Algeiros, combined and their consistency initially checked according to the above data in terms of colour and odour. Any solid proportions present (e.g., wood) are mechanically removed. A chemical-analytical determination of the wax proportion, which should be between 10 and 30% by weight, is additionally carried out.

In a preferred exemplary embodiment the term propolis also includes, in this case, a prepared propolis obtained from the standardised propolis described above by suitable processing. A wax proportion of the prepared propolis is preferably between 15 and 25% by weight. The processing takes place preferably by the use of conventional methods of separating any proteins or other possible allergens present. It is also preferable for the prepared propolis to be subjected to a sterilisation process in which readily volatile constituents are driven off, e.g., by the supply of thermal energy, and chemical reactions could take place inside the propolis, altering the composition of the product.

A prepared propolis within the meaning of the invention is also understood to include a mixture suitable for coating and containing propolis in a proportion by weight of at least 30% by weight, preferably at least 50% by weight, and more preferably, at least 80% by weight. The remaining constituents of such a mixture may include, for example, beeswax, fats, hydrocarbons, fatty acids or the like, whose addition may be appropriate for simplifying the processing of the material, or may bring about improved adaptation of the material properties to a specific implant. It is therefore conceivable, for example, that the viscosity and adhesion of the material may be influenced by the addition of long-chain compounds such as waxes or fats.

It is also conceivable and preferable to use propolis as the carrier matrix for pharmaceutical active ingredients such as paclitaxel or sirolimus. Here it is advantageous for the propolis to be hydrophobic and therefore to be able to dissolve pharmaceutical active ingredients that are difficult to dissolve in water in larger quantities. After implantation the active ingredients gradually penetrate the surrounding tissue by diffusion and develop their effect according to the regulations.

It has been demonstrated that propolis has antibacterial, antifungal, antiviral, tumour cytotoxic, tumour inhibiting, local anaesthetic, anti-inflammatory and spasmolytic properties. Some active components of propolis, to which the above-mentioned effects are ascribed, could already be identified. Therefore: (i) an antibacterial action is ascribed to pinocembrin, galangin, caffeic acid and ferulic acid, (ii) an antifungal action is ascribed to pinocembrin, 3-actyl pinobanksin, caffeic acid, p-cumaric acid benzyl ester, sakuranetin and pterostilben, (iii) an antiviral action is ascribed to caffeic acid and quercetin, a tumour cytotoxic or tumour inhibiting action is ascribed to caffeic acid phenyl ethyl ester (CAE), a local anaesthetic action is ascribed to pinocembrin, pinostrobin and caffeic acid ester, (vi) an anti-inflammatory action is ascribed to caffeic acid and (vii) a spasmolytic action is ascribed to quercetin, kaemperide and pectolinaringenin.

Propolis is easily accessible, can be purchased at low cost, is of a sticky, wax-like consistency at body temperature, and can be easily applied to implant surfaces by conventional process techniques in uniform coverage.

The actions and properties predestine propolis as a coating material for medical implants.

A preferred application of propolis resides in its use as a coating material for pacemakers, defibrillators, cardiac or venous valves or vascular prostheses.

Diseases of the cardiovascular system in particular present the risks that infections may develop out of control, particularly as the processes already described above are promoted in the implantation of pacemakers and defibrillators by the gradual formation of biofilms on the implant, and the patients are already weakened as a result of the disease. Pacemakers and defibrillators are complex electrical appliances which are normally bounded on the outside by a shell. Cardiac or venous valves or vascular prostheses are filigree structures which must meet a plurality of conditions in order to guarantee their functionality. A vascular prosthesis is a hose which replaces or bridges a blood vessel—in most cases an artery. A geometry of the implants and the materials selected for the shells/valves/hoses cannot be altered without difficulty for reducing the risk of rejection or infection. Furthermore, explanation of pacemakers and defibrillators or cardiac and venous valves or vascular prostheses is particularly stressful for the patient because of the symptoms. For this reason coating with propolis is particularly appropriate for pacemakers and defibrillators, cardiac and venous valves.

A further preferred application of propolis resides in its use as a coating material for stents.

Stents are used in 70% of all percutaneous interventions, but in 25% of all cases in-stent re-stenosis takes place, with an attendant rapid neo-intimal growth caused by rapid proliferation of the arterial smooth muscle cells. To reduce the re-stenosis rates a wide variety of approaches was followed, e.g., intercoronary radioactive radiation (brachytherapy), but here peripheral re-stenoses, delayed curing and incomplete endothelialisation occurred. In the last few years stents have been coated with various pharmaceutical active substances, either by direct binding to the stent surface or embedding in a polymer as the carrier matrix. In this case a sufficiently local active substance concentration and distribution in the vessel wall must necessarily be aimed for. Hitherto the stents releasing the active substances sirolimus or paclitaxel have been most successful in reducing the re-stenosis rate. Very recently, however, cases of subacute thromboses and allergic reactions have occurred, probably due to the polymers used for the carrier matrix. In particular, patients with multiple or severe symptoms (such as diabetes, complex lesions, small vessels, long lesions) show increased thrombosis rates in some cases. Non-degradable polymers (e.g., polyurethanes, polymethacrylates), degradable polymers (e.g., polyhydroxybutyric acid, polylactides), synthetic polymers (phosphoryl cholin) or polymers of biological origin (hyaluronic acid) may be used for the stent coating (the carrier matrix). Some of the polymers give rise to strong inflammatory reactions or induce undesirable proliferation. Direct binding of the active substance to the stent surface, without polymer, is therefore resorted to, but this is technically expensive. Moreover, the latter measure can only reduce, or at best eliminate, the risk or re-stenosis after percutaneous transluminar coronary angioplasty (PTCA). The risk of infections is not removed, therefore, and can only be systemically treated in the case of stents because of rapid growing into the surrounding tissue. Here a coating material which has both anti-infectious properties but is also suitable for re-stenosis prophylaxis, according to the initial data obtained by the applicant, can provide a remedy. Propolis is therefore ideal as a coating material for stents.

According to a preferred exemplary embodiment the stent consists wholly or in parts of a biocorrodible metallic alloy, particularly magnesium alloy. Biocorrodible means that the material is gradually degraded after implantation, e.g., by hydrolytic or enzymatic processes. Such alloys are known, for example, from DE 1 419 793 A1, the disclosed content of which is not fully reproduced here in terms of the magnesium alloys used. The use of propolis as a coating material for stents of a biocorrodible metallic alloy, particularly magnesium alloy, is therefore particularly preferred because, as is well known, propolis is hydrophobic and coating the implant with it therefore inhibits/delays the degradation processes. In other words the degradation behaviour of the implant can be controlled by such a hydrophobic coating. If necessary the degradation behaviour may, for example, be specifically influenced at individual points of the implant by specifying different coating thicknesses in different areas of the implant, thereby achieving as uniform a degradation of the implant as possible.

A further aspect of the invention resides in the surprising knowledge that propolis is suitable for the manufacture of a drug for re-stenosis prophylaxis in human coronary arteries after percutaneous transluminar coronary angioplasty (PTCA).

After initial tests conducted by the applicant it was shown, in particular, that the caffeic acid phenylethyl ester (CAPE; CAS 104594-70-9), with the formula (I)

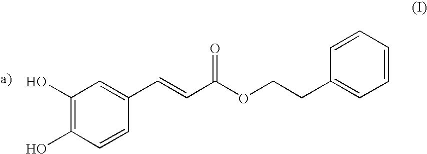

is probably one of the active components and is suitable for re-stenosis prophylaxis in human coronary arteries after percutaneous transluminar coronary angioplasty (PTCA).

In this connection it is known that caffeic acid phenylethyl ester is an inhibitor of the transcription factor F_{K}B (demonstrated, among other things, (i) for human breast cancer cells of the type MCF-7: M. Watabe et al., The Journal of Biological Chemistry. Vol. 279, No. 7, pp. 6017-6026, 2004 and (ii) for T cells: N. Márquez et al., The Journal of Pharmacology and Experimental Therapeutics. Vol. 308, No. 3, pp. 999-1001, 2004). Furthermore, K. Yamasaki et al. describes, in Gene Therapy, Vol. 10, pp. 356-364, 2003, tests on the pig model in which a neo-intimal formation is considerably reduced by the application of a cis element decoy for inhibiting NF_{K}B after balloon angioplasty.

What was now surprising was that propolis 75% inhibited the proliferation of human arterial smooth muscle cells from coronary arteries compared to untreated cells, whilst the vitality and proliferation of human and arterial endothelial cells is only reduced by 30% at the same concentration of the substance. As far as neo-intimal proliferation is concerned, an inhibition of the smooth muscle cells is desirable. On the other hand, the growth of the endothelial cells is important for the endothelialisation. Tests on human arterial endothelial cells for the inhibition of the TNFα induced NF_{K}B activation showed almost complete inhibition of the NF_{K}B activation by propolis (measured as translocation from the p65 sub-unit in the nucleus; evidence with immune fluorescence). According to the present state of knowledge it is assumed that one of the constituents of propolis participating in this cell type specific action mechanism is caffeic acid phenylethyl ester. Propolis and the compound mentioned are therefore particularly suitable for local therapy of the coronary vessel sections affected by PTCA by application of the same, as a coating material, to vascular prostheses or catheters.

A brief description of some of the cell tests follows.

Cells Used

Human arterial endothelial cells (EC's) and human arterial smooth muscle cells (SMC's), commercially available from PromoCell GmbH, Heidelberg, Germany.

Culture Media Used

“Endothelial Cell Growth Medium MV Kit” and “Smooth Muscle Cell Growth Medium 2 Kit”, commercially available from PromoCell GmbH, Heidelberg, Germany.

Cultivation of the Cells

The cells are grown according to the standard instructions normally issued for cell culture technology, and according to the working instructions of Promocell.

Materials Used

Propolis raw material (manufacturer: R. Hesselbarth, Albbruck-Buch, Germany; condition: yellowish-brown, resin-wax like substance with the smell of conifers) was dissolve din 70% ethyl alcohol. After the insoluble constituents were deposited, the residue was filtered and stored in tightly sealable glass containers. The solid content of the extract used for the culture experiments was determined as 450 mg dry substance per ml of extract.

Test Preparation/Cell Cultivation

Initially dilution series of the propolis extract were manufactured (using a 70% aqueous ethanol solution as dilution medium), then added to the cell cultures in such quantity that concentrations of 16.37 μg/ml, 8.175 μg/ml, 4.1 μg/ml and 2.0 μg/mol dry mass per ml of cell culture formulation resulted. Control samples of the solvent were also taken for determining a possible association with an independent influence of the solvent on the proliferation and vitality of the cells.

The above-mentioned diluted propolis formulations were added to the cultures for in vitro testing of the substances 48 hours after sowing of the cells to be tested (96-well plates, 0.3×10⁴ cells/well), and the vitality and proliferation tests (see below) carried out according to the corresponding working instructions of the test manufacturers, immediately after the incubation times indicated below.

Propolis was tested in three parallel formulations in the individual experiments.

Vitality Test (MTS)

After incubating the cells for the different incubation times (12 h, 24 h, 48 h, 72 h at 37° C. and 5% CO₂), the vitality was measured. Here the “Cell Titre 96 Aqueous One Solution Cell Proliferation Assay” from Promega GmbH, Germany, was used. This test is a colorimetric method for determining the metabolic activity of cells.

Proliferation Test (BrdU-ELISA)

The proliferation of the cells was measured after the different incubation times (12 h, 24 h, 48 h, 72 h at 37° C. and 5% CO₂). For this purpose the “Cell Proliferation Elisa BrdU” from Roche Deutschland Holding GmbH, Germany, was used. The measurement was carried out with the “μQuant” Elisa reader from MWG Biotech AG, Germany.

Activation of NF_{K}B

Primary human arterial EC's or SMC's were sown in 24-well plates (1.5×104/well). One day later the cells were incubated with the different dilutions of propolis (16.4 μg/ml, 8.175 μg/ml, 4.1 μg/ml and 2.05 μg/ml cell culture formulation) for one hour and 40 minutes. A further incubation was then carried out for 45 minutes with or without 25 ng/ml TNFα (Sigma-Aldrich Chemie GmbH, Germany). The cells were incubated only with or without 25 ng/ml TNFα. The cells were then washed with PBS, incubated for 20 minutes at 3% PFA, TX-100 permeabilised ((i) Sodeik B, EbersoldMW, Helenius A.: Microtubule-mediated transport of incoming herpes simplex virus 1 capsids to the nucleus. J Cell Biol. 1997 Mar. 10; 136(5): 1007-1021; (ii) Dohner, K. Wolfstein A, Prank U. Echeverri C, Dujardin D. Vallee R, Sodeik B. L: Function of dynein and dynactin in herpes simplex virus capsid transport. Mol Biol Cell. 2002 August; 13(8): 2795-280) and marked with an antibody against NF_{K}B p65 (Zymed). The evidence was provided of the binding of an Alexa546-coupled secondary antibody (Molecular Probes) and fluorescence microscopic evaluation.

Detection and Apoptosis

Primary human arterial EC's or SMC's were own in 24-well plates (1.5×10⁴/well). One day later the cells were incubated for a further 24 h with different dilutions of propolis (16.37 μg/ml, 8.175 μg/ml, 4.1 μg/ml, 2.05 μg/ml). The cells were then washed with PBS, incubated for 20 minutes with 3% PFA and permeabilised with 0.1% TritonX-100. The TUNEL assay was then carried out according to the Roche protocol “In Situ Cell Death Detection Kit” to detect apoptotic cells. Finally a Dapi colour check was carried out. The evaluation was conducted by means of fluorescence microscopy.

Results

Influence of Proposal on the Vitality of EC's and SMC's

The effect of propolis in concentrations of 16.37 μg/ml, 8.175 μg/ml, 4.1 μg/ml and 2.05 μg/ml cell culture formation was examined. In preliminary tests it had been demonstrated that higher concentrations were not suitable for the colorimetric assays both because of the intensive natural colour of propolis and the high cell death inducibility.

The vitality of the human arterial smooth muscle cells (SMC's) and of the endothelial cells (EC's) not only depends on the concentration of propolis but also on the length of the incubation time. The higher the concentration of propolis, the more the vitality of the cells will be inhibited. In this case the inhibition of vitality is greatest in the first 24 hours, after which a weakening of the inhibition may be observed. Moreover, it was established that the EC's are more insensitive to propolis than the SMC's. At the same concentration and incubation time the vitality of the EC's during incubation with propolis was roughly half to a third less inhibited than the vitality of the SMC's.

Influence of Propolis on the Proliferation of EC's and SMC's

The effect of propolis in concentrations of 16.37 μg/ml, 8.175 μg/ml, 4.1 μg/ml and 2.05 μg/ml as examined. In preliminary tests it had also been demonstrated that higher concentrations were not suitable for the colorimetric assays both because of the intensive natural colour of propolis and the high cell death inducibility.

The influence of propolis on the proliferation of SMC's and EC's, like its influence on vitality, depends both on its concentration and on the length of the incubation time. The higher the concentration of propolis and the longer the incubation time, the more the proliferation of the cells is inhibited.

As with vitality, the inhabitation of proliferation is greatest in the first 24 hours, after which the inhibition weakens.

Here too lower sensibility of the EC's to propolis is observed compared to the SMC's. At the same concentration and incubation time the proliferation of the EC's by propolis is inhibited many times less than the proliferation of the SMC's.

Examination for Activation of NF_{K}B

The EC appears to show almost complete inhibition of the TNFα induced NF_{K}B activation by propolis (1:32). Higher dilutions had no inhibitory effect on the TNFα induced NF_{K}B activation. To prevent propolis itself from being NF_{K}B activated, the cells were incubated with propolis without TNFα. At all the concentrations examined, no activation of NF_{K}B activation was observed.

Detection of Apoptotic Cells

A first indication of apoptosis was observed in smooth muscle cells after the addition of propolis in a concentration of 8.175 μg/ml. When 4.1 μg/ml of propolis were added, apoptotic cells were not clearly detected because the signals were too weak. At the lowest concentrate, 2.05 μg/ml of propolis, no apoptic cells were observed.

In the case of endothelial cells no apoptosis was demonstrated at any of the concentrations used.

All patents, applications and publications referred to herein are incorporated by reference in their entirety.

1. An application of propolis as a coating material for medical implants.

2. The application of claim 1, wherein the medical implant is a stent.

3. The application of claim 1, wherein the medical implant is a pacemaker or a defibrillator.

4. The application of claim 1, wherein the medical implant is a cardiac or venous valve.

5. The application of claim 1, wherein the medical implant is a vascular prosthesis.

6. A medical implant with a coating consisting of or containing propolis.

7. An application of propolis for manufacturing a drug for re-stenosis prophylaxis after percutaneous transluminar coronary angioplasty (PTCA) in human coronary arteries.

8. An application of caffeic acid phenylethyl ester for the manufacture of a drug for re-stenosis prophylaxis after percutaneous transluminar coronary angioplasty (PTCA) in human coronary arteries.
